(19) United States
(12) Patent Application Publication
(10) Pub. No.: US 2022/0235400 A1
(43) Pub. Date: Jul. 28, 2022

(54) METHODS FOR DETECTING A LEVEL OF H. PYLORI IN A FECAL SAMPLE

(71) Applicant: AMERICAN MOLECULAR LABORATORIES, INC., Vernon Hills, IL (US)

(72) Inventors: Yi ZHOU, Vernon Hills, IL (US); Hongjun ZHANG, Vernon Hills, IL (US); Rajarao KAKUTURU, Vernon Hills, IL (US); Zhaozhong CHONG, Vernon Hills, IL (US)

(21) Appl. No.: 17/613,737

(22) PCT Filed: May 26, 2020
(86) PCT No.: PCT/US2020/034508

§ 371 Date: Nov. 23, 2021

Related U.S. Application Data
(60) Provisional application No. 62/852,016, filed on May 23, 2019.

(51) Int. Cl. C12Q 1/689; C12Q 1/6806

(52) U.S. Cl. CPC C12Q 1/689; C12Q 2600/118; C12Q 1/6806

(57) ABSTRACT

The present disclosure provides methods and materials for detecting a level of H. pylori in a sample.

### CROSS-REFERENCE TO RELATED APPLICATIONS

This application claims the benefit of and priority to U.S. Patent Application No. 62/852,016, filed May 23, 2019, which is incorporated herein by reference in its entirety.

### FIELD

The present disclosure generally relates to methods and materials for detection of Helicobacter pylori (H. pylori).

### SEQUENCE LISTING

The instant application contains a Sequence Listing which has been submitted electronically in ASCII format and is hereby incorporated by reference in its entirety. Said ASCII copy, created on May 26, 2020, is named “116110-5004-WO_ST25_Sequence_Listing” and is 4 kilobytes in size.

### BACKGROUND

H. pylori is one of the most prevalent global human pathogens that infects an estimated 50% of the world's population. H. pylori is primarily found in the stomach and plays an important role in the pathogenesis of chronic gastritis, peptic ulcers, mucosa-associated lymphoid tissue (MALT) lymphoma, gastric carcinoma, and gastric cancer.

Current diagnostic tests for H. pylori, including immunohistochemistry (IHC), urea breath test (UBT), and stool antigen tests, are disfavored for various reasons. IHC requires invasive gastric biopsy, and is particularly disfavored for follow-up after treatment. Both UBT and stool antigen tests lack fidelity, producing unacceptable false positive and negative rates. Furthermore, treatment with proton pump inhibitors, a common regimen for patients exhibiting symptoms associated with H. pylori infection, can affect both UBT and stool antigen results, complicating their interpretation. Moreover, UBT cannot be used for children under 3-years old, or for pregnant women.

Traditional methods of detecting H. pylori have other disadvantages. For example, such methods are only capable of testing a single H. pylori strain and thus may fail to provide a complete picture of the population of different strains in a sample. This is particularly true in regions with high H. pylori infection rates where patients are more likely to be infected with multiple strains of H. pylori. Additionally, these methods require culturing H. pylori, which is tedious and has a high frequency of failure due to sampling bias and poor sample preservation during shipment.

Thus, there is a need for a faster, more reliable, non-invasive test to determine the presence of H. pylori in a patient sample.

### SUMMARY

The present disclosure relates to methods and materials for detection of Helicobacter pylori (H. pylori) in a sample (e.g., fecal sample) including, for example, detection of a threshold level of H. pylori in a sample from a subject. In embodiments, the disclosure further relates to detecting a DNA segment from a member of the Bacteroides genus (a ubiquitous genus of gut bacteria in normal subjects) and using a level of the Bacteroides DNA segment as an internal control to determine a level of H. pylori in a fecal sample. Additionally, the present disclosure relates to compositions and kits comprising PCR primer pairs for multiplexed, quantitative PCR of H. pylori DNA and Bacteroides DNA from a fecal sample.

The present disclosure also provides methods and materials for detecting a level of H. pylori present in a fecal sample. The methods may comprise: obtaining a fecal sample from a subject, extracting H. pylori and Bacteroides DNA from the fecal sample, amplifying one or more H. pylori DNA segments and one or more Bacteroides DNA segments, detecting an amount of the one or more H. pylori DNA segments and an amount of the one or more Bacteroides DNA segments, and comparing the amount of the one or more H. pylori DNA segments to the amount of the one or more Bacteroides DNA segments to determine the level of H. pylori in the fecal sample.

In some embodiments of each or any of the above or below mentioned embodiments, the disclosure provides methods and materials for detecting the level of H. pylori present in a fecal sample and determining whether the fecal sample is H. pylori positive, H. pylori weakly positive, or H. pylori negative.

In some embodiments of each or any of the above or below mentioned embodiments, the disclosure provides a method of determining that a fecal sample is H. pylori positive if a threshold level of about 5 or more copies of one or more H. pylori DNA segments is detected, and a level of about 50, about 60, about 70, about 80, about 90, about 100, about 110, about 120, about 130, about 140, about 150, or more (preferably 100) copies one or more Bacteroides DNA segments is detected.

In some embodiments of each or any of the above or below mentioned embodiments, the disclosure provides a method of determining that the fecal sample is H. pylori weakly positive if a threshold level of about 2 to about 5 copies of one or more H. pylori DNA segments is detected, and a level of about 50, about 60, about 70, about 80, about 90, about 100, about 110, about 120, about 130, about 140, about 150, or more (preferably 100) copies one or more Bacteroides DNA segments is detected.

In some embodiments of each or any of the above or below mentioned embodiments, the disclosure provides a method of determining that the fecal sample is H. pylori negative if a threshold level of less than about 2 copies of one or more H. pylori DNA segments is detected, and a level of about 50, about 60, about 70, about 80, about 90, about 100, about 110, about 120, about 130, about 140, about 150, or more (preferably 100) copies one or more Bacteroides DNA segments is detected.

In some embodiments of each or any of the above or below mentioned embodiments, the disclosure provides a method of determining a level of H. pylori in a fecal sample, wherein one or more H. pylori DNA segments and one or more Bacteroides DNA segments are amplified by quantitative PCR, and wherein the amount of the one or more H. pylori DNA segments and the amount of the one or more Bacteroides DNA segments is detected using a probe sequence during a quantitative PCR reaction. In further embodiments, the quantitative PCR reaction is multiplexed to detect the amount of the one or more H. pylori DNA segments and the amount of the one or more Bacteroides DNA segments present in a sample in a single quantitative PCR reaction.

In some embodiments of each or any of the above or below mentioned embodiments, the disclosure provides a method of determining a level of H. pylori in a fecal sample comprising amplifying one or more H. pylori DNA segments, wherein the one or more H. pylori DNA segments are evolutionarily conserved.

In some embodiments of each or any of the above or below mentioned embodiments, the disclosure provides a method of determining a level of H. pylori in a fecal sample comprising amplifying one or more H. pylori DNA segments, wherein the one or more H. pylori DNA segments comprise an H. pylori 23 S rRNA gene, an H. pylori 16S rRNA gene, and an H. pylori Urease A gene. In some embodiments, the methods of the disclosure comprise amplifying two of more of the H. pylori 23S rRNA gene, H. pylori 16S rRNA gene, or H. pylori Urease A gene. In still further embodiments, the methods of the disclosure comprise amplifying each of the H. pylori 23S rRNA gene, H. pylori 16S rRNA gene, and H. pylori Urease A gene.

In some embodiments of each or any of the above or below mentioned embodiments, the disclosure provides a method of determining a level of a Bacteroides DNA segment, wherein the Bacteroides DNA segment comprises one or more DNA segments from Bacteroides fragilis, Bacteroides melaninogenicus, Bacteroides oralis, or a combination thereof. In some embodiments, the methods of the disclosure comprise detecting one or more Bacteroides DNA segments from a Bacteroides species that is present in a human regardless of age and condition.

In some embodiments of each or any of the above or below mentioned embodiments, amplifying the one or more H. pylori DNA segments and the one or more Bacteroides DNA segments by quantitative PCR comprises selecting PCR primer pairs for producing amplicons comprising the one or more H. pylori DNA segments, selecting PCR primer pairs for producing amplicons comprising the one or more Bacteroides DNA segments, and segregating PCR primer pairs comprising one or more primers that interfere with amplicon generation by another PCR primer pair into separate PCR primer pair pools, wherein each of the separate PCR primer pair pools contain a plurality of PCR primer pairs.

In some embodiments of each or any of the above or below mentioned embodiments, amplifying the one or more H. pylori DNA segments in a PCR reaction comprises using one or a plurality of PCR primer pairs selected from SEQ ID NO: 1, SEQ ID NO: 2, SEQ ID NO: 4, SEQ ID NO: 5, SEQ ID NO: 7 and SEQ ID NO: 8. In further embodiments, H. pylori DNA segments are amplified in a quantitative PCR reaction comprising one or a plurality of probe sequences selected from SEQ ID NO: 3, SEQ ID NO: 6 and SEQ ID NO: 9.

In some embodiments of each or any of the above or below mentioned embodiments, amplifying the one or more Bacteroides DNA segments in a PCR reaction comprises using one or a plurality of PCR primer pairs selected from [SEQ IDs]. In further embodiments, Bacteroides DNA segments are amplified in a quantitative PCR reaction comprising one or a plurality of probe sequences selected from SEQ ID NO: 10 and SEQ ID NO: 11.

In some embodiments of each or any of the above or below mentioned embodiments, determining a level of H. pylori in a fecal sample comprises obtaining between about 0.5 grams and about 1.0 grams of fecal matter from a subject. In some embodiments, DNA is extracted from a fecal sample by bead homogenizing the fecal sample in a lysis buffer, wherein the lysis buffer comprises ingredients capable of breaking a bacterial cell wall, digesting protein, denaturing protein, dispersing fat, precipitating polysaccharides, or a combination thereof.

In some embodiments of each or any of the above or below mentioned embodiments, the disclosure provides methods of extracting DNA from a fecal sample comprises loading the homogenized, lysed fecal sample onto a filter column comprising a filter and a silica membrane, wherein the filter column is housed within a collection vial with a closed bottom and an open top for receiving the filter column, forcing the soluble contents of the homogenized, lysed fecal samples through the silica membrane, and eluting the DNA from the silica membrane.

In some embodiments of each or any of the above or below mentioned embodiments, the disclosure provides compositions for determining the presence of H. pylori in a sample comprising, one or a plurality of PCR primer pairs that amplify one or more H. pylori DNA segments, and one or a plurality of control PCR primer pairs that amplify one or more Bacteroides DNA segments. In further embodiments, compositions of the disclosure comprise PCR primer pairs that amplify one or more conserved H. pylori DNA segments.

In some embodiments of each or any of the above or below mentioned embodiments, the disclosure provides compositions comprising PCR primer pairs that amplify one or more H. pylori DNA segments comprising an H. pylori 23S rRNA gene, an H. pylori 16S rRNA gene, or an H. pylori Urease A gene. In embodiments, disclosure provides compositions comprising PCR primer pairs that amplify each of an H. pylori 23S rRNA gene, an H. pylori 16S rRNA gene, and an H. pylori Urease A gene. In certain embodiments, the disclosure provides PCR primer pairs comprising SEQ ID NO: 1, SEQ ID NO: 2, SEQ ID NO: 4, SEQ ID NO: 5, SEQ ID NO: 7 and SEQ ID NO: 8.

In some embodiments of each or any of the above or below mentioned embodiments, the disclosure provides compositions comprising PCR primer pairs that amplify one or more Bacteroides DNA segments, wherein the Bacteroides DNA segment is from a Bacteroides species that is present in a human regardless of age and condition. In further embodiments, Bacteroides DNA segment is from one or more of DNA segments from Bacteroides fragilis, Bacteroides melaninogenicus, Bacteroides oralis, or a combination thereof.

The present disclosure also provides compositions for determining the presence of H. pylori in a sample comprising, one or a plurality of PCR primer pairs that amplify one or more H. pylori DNA segments, and one or a plurality of control PCR primer pairs that amplify one or more Bacteroides DNA segments, wherein the PCR primer pairs and control PCR primer pairs are capable of being multiplexed. In some embodiments, the PCR primer pairs and control PCR primer pairs are capable of multiplexed quantitative PCR.

In some embodiments of each or any of the above or below mentioned embodiments, the disclosure further comprises a PCR reaction buffer, di-nucleotide triphosphates, and one or more polymerase enzymes.

The present disclosure also provides a kit for detecting the presence of H. pylori in a sample (e.g., a fecal sample) comprising, one or a plurality of PCR primer pairs that amplify one or more H. pylori DNA segments, and one or a plurality of PCR primer pairs that amplify one or more Bacteroides DNA segments. In further embodiments, a kit of the disclosure comprises one or a plurality of probe sequences hybridizing with an amplification product of the one or more H. pylori DNA segments, and one or a plurality of probe sequences hybridizing with an amplification product of the one or more Bacteroides DNA segments. In still further embodiments, a kit of the disclosure comprises a bead homogenization suspension in a lysis buffer, wherein the lysis buffer comprises ingredients capable of breaking a bacterial cell wall, digesting protein, denaturing protein, dispersing fat, precipitating polysaccharides, or a combination thereof. In yet further embodiments, a kit of the disclosure comprises a silica purification reagent and a DNA binding buffer. In some embodiments, a kit of the disclosure further comprising a filter column comprising a filter and a silica membrane, wherein the filter column is housed within a collection vial with a closed bottom and an open top for receiving the filter column, and a wash buffer and an elution buffer. In embodiments, a kit of the disclosure comprises a PCR reaction buffer, di-nucleotide triphosphates, and one or more polymerase enzymes.

The present disclosure also provides methods of H. pylori treatment in a subject comprising obtaining a fecal sample from a subject, extracting H. pylori and Bacteroides DNA from the fecal sample, amplifying one or more H. pylori DNA segments and one or more Bacteroides DNA segments, detecting an amount of the one or more H. pylori DNA segments and an amount of the one or more Bacteroides DNA segments, comparing the amount of the one or more H. pylori DNA segments to the amount of the one or more Bacteroides DNA segments to determine the level of H. pylori in the fecal sample, and administering an H. pylori treatment if H. pylori is present at a threshold level. In some embodiments, the disclosure provides methods of H. pylori treatment in a subject, wherein treatment is administered if about 5 or more copies of an H. pylori DNA segment are detected in a fecal sample from a subject and about 50, about 60, about 70, about 80, about 90, about 100, about 110, about 120, about 130, about 140, about 150, or more (preferably 100) copies one or more Bacteroides DNA segments is detected.

The present disclosure also provides methods of monitoring H. pylori treatment in a subject comprising obtaining a fecal sample from the subject after administering an H. pylori treatment, extracting H. pylori and Bacteroides DNA from the fecal sample, amplifying one or more H. pylori DNA segments and one or more Bacteroides DNA segments, detecting an amount of the one or more H. pylori DNA segments and an amount of the one or more Bacteroides DNA segments, and comparing the amount of the one or more H. pylori DNA segments to the amount of the one or more Bacteroides DNA segments to determine the level of H. pylori in the fecal sample. In some embodiments, the disclosure provides methods of monitoring H. pylori treatment in a subject, wherein the fecal sample is collected at least four weeks after treatment. In some embodiments, the disclosure provides methods of monitoring H. pylori treatment in a subject, wherein the method is repeated daily, weekly, monthly, or yearly.

### BRIEF DESCRIPTION OF THE DRAWINGS

FIG. 1 is a stained agarose gel electrophoresis analysis of PCR products comprising: lane 1, DNA standard ladder; lane 2, Helicobacter fennelliae (DSM7491), lane 3, Helicobacter cinaedi (DSM5359), lane 4, Campylobacter jejuni subsp jejuni (DSM4688), lane 5, Lactobacillus reuteri (DSM20016), lane 6, Streptococcus suis (DSM9682), lane 7, Helicobacter pylori (HP26695), lane 8, no template control, lane 9, DNA Ladder.

FIG. 2 is a stained agarose gel electrophoresis analysis of PCR amplification products using the indicated template copy numbers: lane 1, DNA standard ladder, lane 2, 10,000 copies, lane 3, 1,000 copies, lane 4, 100 copies, lane 5, 10 copies, lane 6, 2 copies, lane 7, no template control. Conventional PCR and agarose gel electrophoresis does not detect 2 copies or less H. pylori.

FIG. 3 shows a column for fecal DNA extraction.

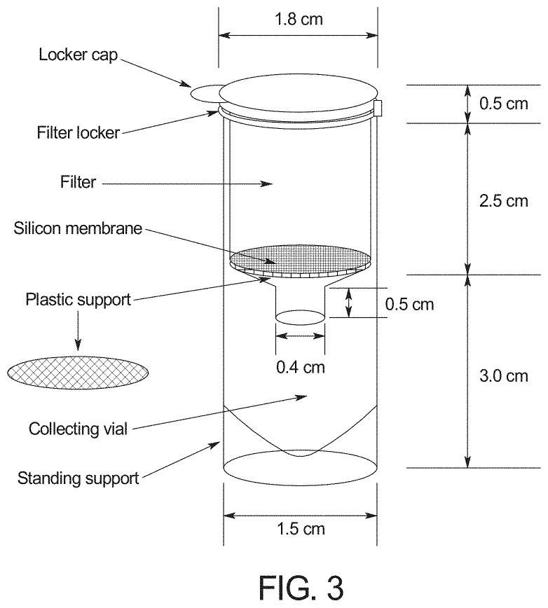

### DETAILED DESCRIPTION

The present disclosure relates to methods and materials for detection of Helicobacter pylori (H. pylori) in a fecal sample including, for example, detecting a threshold level of H. pylori in a sample of fecal matter from a subject. In embodiments, the disclosure further relates to detecting a DNA segment from a member of the Bacteroides genus, a ubiquitous genus of gut bacteria in normal subjects, and using the level of the Bacteroides DNA segment as an internal control in a multiplexed quantitative PCR reaction to accurately determine the level of H. pylori in a fecal sample. Additionally, the present disclosure relates to compositions and kits comprising PCR primer pairs for multiplexed, quantitative PCR of H. pylori DNA and Bacteroides DNA from a fecal sample. Thus, embodiments of the disclosure provide methods, compositions, and kits that surprisingly permit the detection of as few as about 2 copies of an H. pylori DNA segment in a fecal sample.

In some embodiments, the methods of the disclosure further comprise determining the presence and relative amount of H. pylori in a fecal sample. For example, the disclosed methods determine whether a fecal sample is H. pylori positive, H. pylori weakly positive, or H. pylori negative.

Embodiments of the disclosure generally involve obtaining a fecal sample from a subject, extracting H. pylori and Bacteroides DNA from the fecal sample, amplifying one or more H. pylori DNA segments and one or more Bacteroides DNA segments, detecting an amount of the one or more H. pylori DNA segments and an amount of the one or more Bacteroides DNA segments, and comparing the amount of the one or more H. pylori DNA segments to the amount of the one or more Bacteroides DNA segments to determine the level of H. pylori in the fecal sample. In some embodiments, the disclosure further comprises monitoring the formation of PCR amplification products comprising H. pylori DNA and Bacteroides DNA segments using real time amplification detection systems, and quantifying the amount of the DNA segments in the sample.

Where the term “comprising” is used in the present description and the claims, it does not exclude other elements or steps. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

In case, numerical values are indicated in the context of the present disclosure the skilled person will understand that the technical effect of the feature in question is ensured within an interval of accuracy, which typically encompasses a deviation of the numerical value given of ±10%, and preferably of ±5%.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventor for carrying out the disclosure. Of course, variations on these described embodiments will become apparent to those of ordinary skill in the art upon reading the foregoing description. The inventor expects skilled artisans to employ such variations as appropriate, and the inventor intends for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

Further definitions of terms will be given in the following in the context of which the terms are used. The following terms or definitions are provided solely to aid in the understanding of the invention. These definitions should not be construed to have a scope less than understood by a person of ordinary skill in the art.

As used herein, a “sample” or “fecal sample” or “stool sample” means a sample of feces collected from a subject. A sample may be directly tested or else all or some of the nucleic acid present in the sample may be isolated prior to testing. In yet another example, the sample may be partially purified or otherwise enriched prior to analysis. For example, to the extent that a sample comprises a very diverse cell population, it may be desirable to enrich for a sub-population of particular interest. It is within the scope of the present invention for the target cell population or molecules derived therefrom to be treated prior to testing, for example, inactivation of live virus. It should also be understood that the sample may be freshly harvested or it may have been stored (for example by freezing) prior to testing or otherwise treated prior to testing (such as by undergoing culturing).

As used herein, H. pylori means any of the H. pylori strains known in the art, including for example the strains listed in Table 1.

TABLE 1
H. pylori Strains for Multiplex real time PCR

| NO | H. pylori Strain Name |
|---|---|
| 1 | Helicobacter pylori 2017 |
| 2 | Helicobacter pylori 2018 |
| 3 | Helicobacter pylori 26695 |
| 4 | Helicobacter pylori 35A |
| 5 | Helicobacter pylori 51 |
| 6 | Helicobacter pylori 52 |
| 7 | Helicobacter pylori 83 |
| 8 | Helicobacter pylori 908 |
| 9 | Helicobacter pylori Aklavik117 |
| 10 | Helicobacter pylori Aklavik86 |
| 11 | Helicobacter pylori B38 |
| 12 | Helicobacter pylori B8 |
| 13 | Helicobacter pylori BM012A |
| 14 | Helicobacter pylori BM012S |
| 15 | Helicobacter pylori Cuz20 |
| 16 | Helicobacter pylori ELS37 |
| 17 | Helicobacter pylori F16 |
| 18 | Helicobacter pylori F30 |
| 19 | Helicobacter pylori F32 |
| 20 | Helicobacter pylori F57 |
| 21 | Helicobacter pylori G27 |
| 22 | Helicobacter pylori Gambia94/24 |
| 23 | Helicobacter pylori HPAG1 |
| 24 | Helicobacter pylori HUP-B14 |
| 25 | Helicobacter pylori India7 |
| 26 | Helicobacter pylori Lithuania75 |
| 27 | Helicobacter pylori OK113 DNA |
| 28 | Helicobacter pylori OK310 |
| 29 | Helicobacter pylori P12 |
| 30 | Helicobacter pylori PeCan18 |
| 31 | Helicobacter pylori PeCan4 |
| 32 | Helicobacter pylori Puno120 |
| 33 | Helicobacter pylori Puno135 |
| 34 | Helicobacter pylori Rif1 |
| 35 | Helicobacter pylori Rif2 |
| 36 | Helicobacter pylori SJM180 |
| 37 | Helicobacter pylori SNT49 |
| 38 | Helicobacter pylori Sat464 |
| 39 | Helicobacter pylori Shi112 |
| 40 | Helicobacter pylori Shi169 |
| 41 | Helicobacter pylori Shi417 |
| 42 | Helicobacter pylori Shi470 |
| 43 | Helicobacter pylori SouthAfrica20 |
| 44 | Helicobacter pylori SouthAfrica7 |
| 45 | Helicobacter pylori UM032 |
| 46 | Helicobacter pylori UM037 |
| 47 | Helicobacter pylori UM066 |
| 48 | Helicobacter pylori UM298 |
| 49 | Helicobacter pylori UM299 |
| 50 | Helicobacter pylori XZ274 |
| 51 | Helicobacter pylori v225d |
| 52 | Helicobacter pylori-strain J99 |
| 53 | Helicobacter pylori HPbs1 |
| 54 | Helicobacter pylori TH2099 |
| 55 | Helicobacter pylori GD63 |
| 56 | Helicobacter pylori HP14039 |
| 57 | Helicobacter pylori HPbs3 |
| 58 | Helicobacter pylori HPbs2 |
| 59 | Helicobacter pylori PMSS1 |
| 60 | Helicobacter pylori ATCC 43504 |
| 61 | Helicobacter pylori H-137 |
| 62 | Helicobacter pylori NCTC12813 |
| 63 | Helicobacter pylori NCTC13345 |
| 64 | Helicobacter pylori NCTC11637 |
| 65 | Helicobacter pylori FDAARGOS 298 |
| 66 | Helicobacter pylori 7.13 R1B |
| 67 | Helicobacter pylori B128 1 |
| 68 | Helicobacter pylori 7.13 D3c |
| 69 | Helicobacter pylori 7.13 D2b |
| 70 | Helicobacter pylori 26695 dR |
| 71 | Helicobacter pylori dRdM2addM2 |
| 72 | Helicobacter pylori dRdM1 |
| 73 | Helicobacter pylori HPJP26 |
| 74 | Helicobacter pylori G272 |
| 75 | Helicobacter pylori PMSS1 |
| 76 | Helicobacter pylori 7.13 D3b |
| 77 | Helicobacter pylori 7.13 D3a |
| 78 | Helicobacter pylori 7.13 D2c |
| 79 | Helicobacter pylori HP42k |
| 80 | Helicobacter pylori FDAARGOS 300 |
| 81 | Helicobacter pylori 7.13 R1c |
| 82 | Helicobacter pylori 7.13 R1a |
| 83 | Helicobacter pylori 7.13 R2c |
| 84 | Helicobacter pylori 7.13 R2a |
| 85 | Helicobacter pylori 7.13 R2b |
| 86 | Helicobacter pylori 7.13 D2a |

As used herein, a “DNA segment” comprises a sequence of DNA, such as a gene, a non-coding region, an intron, an exon, or any combination thereof. In some cases, a DNA segment is amplified to generate an amplicon. Reference to a DNA segment should be understood as a reference to a specific section of genomic DNA, such as bacterial genomic DNA. These DNA segments are specified either by reference to a gene name or a set of chromosomal coordinates. Both the gene names and the chromosomal coordinates would be well known to, and understood by, the person of skill in the art.

As used herein, a “threshold level” means a level (e.g., number of copies) of an H. pylori DNA segment that when met or exceeded result in a determination that a sample (e.g., a fecal sample) is positive for the presence of H. pylori.

As used herein, a “probe sequence” is a nucleic acid capable of binding to a target nucleic acid of complementary sequence through one or more types of chemical bonds, usually through complementary base pairing, usually through hydrogen bond formation, thus forming a duplex structure. The probe binds or hybridizes to a “probe binding site.” A probe may include natural (i.e. A, G, C, or T) or modified bases (7-deazaguanosine, inosine, etc.). A probe can be a single stranded oligonucleotide. Oligonucleotide probes can be synthesized or produced from naturally occurring polynucleotides. In addition, the bases in a probe can be joined by a linkage other than a phosphodiester bond, so long as it does not interfere with hybridization.

As used herein, “quantitative PCR” or “qPCR” or “quantitative real time PCR” refers to methods of monitoring the amplification of a DNA segment in a sample in real time to determine the level of the DNA segment in the sample.

In embodiments, the methods of the disclosure comprise obtaining a fecal sample from a subject and extracting DNA from the sample. Feces of any animal can be tested in various embodiments disclosed herein. Samples may be collected by any readily available means, e.g., at a point of care facility by medical professionals, or a by the subject using an at home collection kit. In embodiments, samples are kept refrigerated until testing. In embodiments, preparation of the fecal sample can be accomplished using any of the known methods in the art. For example the soluble portion of the sample can be collected using filtration, centrifugation, or simple mixing followed by gravimetric settling.

Fecal samples can be taken and prepared in many ways. For example, in some embodiments the fecal sample comprises a stool supernatant prepared from a stool homogenate. In some embodiments, the methods comprise exposing the fecal sample to a condition that denatures proteins and nucleic acids before extracting bacterial DNA. For example, some embodiments provide that the condition that denatures nucleic acids comprises heating at 90° C. for 10 minutes.

In some embodiments, the fecal sample is lysed to extract its DNA content in a buffer formulated with an amount of Tris-HCl buffer, ethylenediaminetetraacetic acid (EDTA), NaCl, cetyl trimethylammonium bromide, polyvinyl pyrrolidone, and proteinase. In some embodiments, the DNA extracted from the lysed sample is bound to an affinity reagent (e.g. silica) in a binding buffer comprising an amount of Tris-HCl, EDTA, and guanidine thiocyanate. In some embodiments, the DNA is serially washed in one or more buffers comprising Tris-HCl, EDTA, and ethanol, and eluted from the affinity reagent using an appropriate elution buffer.

Several methods exist for the isolation of DNA from bacterial cells. These methods essentially utilize the same basic procedure. In an exemplary embodiment, bacterial cells in a fecal sample are lysed enzymatically (i.e., lysozyme treatment), mechanically (i.e., bead homogenization) or by repeated freeze-thaw cycles, or combinations of these, followed by dissolution of the cell membrane with alkali and detergents such as sodium dodecyl sulfate (SDS) (Maniatis et al., 1989; Tsai et al., Appl. Environ. Microbiol., 57:1070-1074, 1991; Bej et al., Appl. Environ, Microbiol., 57:1013-1017, 1991). The cell lysate is then treated with proteinases and hexadecyltrimethyl ammonium bromide (CTAB) to degrade proteins and precipitate carbohydrates, respectively. The most common proteinase used in this procedure is proteinase K.

After extraction and physical separation of the DNA from other cellular components (lipid, carbohydrates, proteins), the DNA is isolated, or purified, according to methods known in the art. In certain embodiments, the DNA is isolated by a silica-based method, wherein the DNA is bound to a silica substrate, such as a silica membrane of silica beads, washed, and then eluted in isolated or purified form. In alternative embodiments, the DNA is isolated by phenol/chloroform extraction.

In some embodiments, the disclosure provides methods of extracting DNA from large quantities of fecal matter to enable detection of bacterial species present in low copy number. For example, methods are provided for isolating DNA from between about 0.5 g to about 1.0 g of fecal matter, and detecting a level of H. pylori present in the sample in as low as about 2 to about 5 copy numbers. In other embodiments, DNA is isolated from between about 0.01 g to about 0.1 g, about 0.1 g to about 0.5 g, between about 1.0 g to about 2 g of fecal matter. In some embodiments, the disclosure provides methods for detecting a level of H. pylori present in the sample in as low as about 2 copies, or as high as about 10 copies, about 15 copies, about 20 copies, or greater than 20 copies. In some embodiments, the disclosure provides methods for extracting total DNA present in a fecal sample.

In certain embodiments, the disclosure further provides methods of extracting DNA from a fecal sample comprising loading the homogenized, lysed fecal sample onto a filter column comprising a filter and a silica membrane, wherein the filter column is housed within a collection vial with a closed bottom and an open top for receiving the filter column, forcing the soluble contents of the homogenized, lysed fecal samples through the silica membrane, and eluting the DNA from the silica membrane.

In some embodiments, the disclosure provides methods for detecting an amount of H. pylori present in a fecal sample comprising amplifying one or more H. pylori DNA segments and one or more Bacteroides DNA segments by quantitative PCR, detecting an amount of the one or more H. pylori DNA segments and an amount of the one or more Bacteroides DNA segments, and comparing the amount of the one or more H. pylori DNA segments to the amount of the one or more Bacteroides DNA segments to determine the level of H. pylori in the fecal sample. In embodiments, amplifying a DNA segment comprises quantitative PCR, wherein amplifying the DNA segment is monitored in real time.

For those methods in which the formation of amplification products is monitored, the amplification products can be monitored using any of a variety of real time amplification methods. For example, certain methods involve monitoring the formation of amplification products directly using labels which bind to the amplification product to form a complex that creates a detectable signal. Alternatively, the formation of amplification products can be monitored using probes which are complementary to the amplification products. During the extension phase of the amplification process, alteration of the probe generates a detectable signal which correlates with the formation of amplification product. Fluorogenic nuclease assays such as the “TaqMan” assay (Thermo Fisher) exemplify this type of approach, wherein a probe is used to monitor amplification product formation.

In some embodiments, the methods comprise amplifying an H. pylori 23SrRNA gene, an H. pylori 16S rRNA gene, or an H. pylori Urease A gene. In certain embodiments, each of the 23S rRNA gene, 16S rRNA gene, and Urease A gene are amplified. Accordingly, the disclosure provides methods for multiplexed quantitative amplification, wherein a plurality of DNA segments are amplified and monitored simultaneously. Methods of multiplex quantitative PCR are known in the art, and include use of multiple fluorophores (e.g., FAM, VIC, Cy3).

In some embodiments, each pair of PCR primers targeting a particular DNA segment are segregated into separate PCR primer pair pools containing one or more unique primer pairs targeting different DNA segments. Thus, PCR amplification of each pool produces amplicons specific to the plurality of DNA segments within each pool and minimizes the chance of PCR amplification artifacts such as primer-dimers or cross pair amplicon truncation caused by homologous pairing within overlapping amplicon sequences.

In some embodiments, the disclosure comprises identifying PCR primer pairs suitable for producing amplicons comprising one or more regions of the H. pylori DNA, including identifying PCR primer pairs suitable for producing amplicons comprising one or more H. pylori DNA segments for H. pylori strains known to infect people (e.g., two or more strains known to infect people). In certain embodiments, the disclosure provides PCR primer pairs for amplifying H. pylori DNA segments provided in Table 2. Thus, in embodiments the disclosure provides a method of amplifying an H. pylori DNA segment comprising 23S rRNA using the primer pair comprising SEQ ID NO: 1 and SEQ ID NO: 2. Thus, in other embodiments, the disclosure provides a method of amplifying an H. pylori DNA segment comprising 16S rRNA using the primer pair comprising SEQ ID NO: 4 and SEQ ID NO: 5. In yet other embodiments, the disclosure provides a method of amplifying an H. pylori DNA segment comprising the Urease A gene using the primer pair comprising SEQ ID NO: 7 and SEQ ID NO: 8.

TABLE 1

| | Gene/ | SEQ ID | |
|---|---|---|---|
| Bacteria | Region | NO: | Primers/Probe |
| H. pylori | 23S rRNA | 1 | Forward: TTCATACCAGCGTTGAAGGT |
| | | | (SEQ ID NO: 1) |
| | | 2 | Reverse: TTACGGCGGATACAATTCTCATA |
| | | | (SEQ ID NO: 2) |
| | | 3 | Probe: ATGCTCACTTCATACCGCTCCAGC |
| | | | (SEQ ID NO: 3) |
| H. pylori | 16S rRNA | 4 | Forward: AGACTAAGCCCTCCAACAAC |
| | | | (SEQ ID NO: 4) |
| | | 5 | Reverse: CGACCTGCTGGAACATTAC |
| | | | (SEQ ID NO: 5) |
| | | 6 | Probe: CAGCTTTCGCGCAATCAGCGTCA |
| | | | (SEQ ID NO: 6) |
| H. pylori | Urease A | 7 | Forward: GAAGAAGCGAGAGCTGGTAAA |
| | | | (SEQ ID NO: 7) |
| | | 8 | Reverse: CATCAGGAAACATCGCTTCAATAC |
| | | | (SEQ ID NO: 8) |
| | | 9 | Probe: TGAATTGATGCAAGAAGGGCG |
| | | | (SEQ ID NO: 9) |
| Bacteroides | Conservatie | 10 | Forward: CAGCAGCCGCGGTAATA |
| | Region | | (SEQ ID NO: 10) |
| | | 11 | Reverse: CGCAAACTTTCACAACTGACT |
| | | | (SEQ ID NO: 11) |
| | | 12 | Probe: TAAAGGGAGCGTAGGTGGACTGGTA |
| | | | (SEQ ID NO: 12) |

In further embodiments, the disclosure provides methods of detecting an amount of H. pylori in a fecal sample comprising amplifying a control DNA segment that is ubiquitous in fecal samples (e.g., fecal samples from a majority including, for example, 50%, 60%, 70%, 80%, 90%, or greater of subjects from the same species such as Bacteroides or Homo sapiens) regardless of the age or condition of the subject. In some embodiments, a control DNA segment from the bacterial genus Bacteroides is amplified. In certain embodiments, one or more Bacteroides DNA segments are amplified in a multiplex quantitative PCR reaction with one or more H. pylori DNA segments. Thus, the disclosure further comprises identifying PCR primer pairs suitable for producing amplicons of Bacteroides DNA; identifying PCR primer pairs suitable for producing amplicons comprising the one or more regions of multiple Bacteroides species present in fecal samples of subjects regardless of age and condition. In certain embodiments, one or more Bacteroides DNA segments are amplified from Bacteroides fragilis, Bacteroides melaninogenicus, Bacteroides oralis, or a combination thereof. In particular embodiments, a Bacteroides DNA segment is amplified using a PCR primer pair comprising SEQ ID NO: 10 and SEQ ID NO: 11.

In further embodiments of each or any of the above or below mentioned embodiments, the disclosure provides one or more probes for use in a multiplexed quantitative PCR reaction to detect a level of one or more H. pylori DNA segments and one or more Bacteroides DNA segments. In particular embodiments, the disclosure provides a probe for monitoring amplification of an H. pylori 23s rRNA DNA segment comprising SEQ ID NO: 3, a probe for monitoring amplification of an H. pylori 16s rRNA DNA segment comprising SEQ ID No: 6, a probe for monitoring amplification of a Urease A DNA segment comprising SEQ ID No: 9, and a probe for monitoring amplification of an Bacteroides DNA segment comprising SEQ ID No: 12.

In embodiments, the disclosure provides PCR primer pairs that amplify an H. pylori DNA segment in a quantitative PCR reaction without amplifying other bacterial species present in the sample. In this manner, the disclosed methods do not detect or cross-amplify non-specific bacterial DNA, including Helicobacter fennelliae, Helicobacter cinaedi, Lactobacillus reuteri, Streptococcus suis and Campylobacter jejuni, species normally found in the gut. In an exemplary embodiment, Table 2 shows a PCR primer pair that specifically amplifies an H. pylori DNA segment comprising the 23S rRNA gene, but not amplifying a host of common bacteria present in the gut and feces (Table 3).

TABLE 3

| Sample Name | Target Name | CT Value |
|---|---|---|
| Helicobacter fennelliae (DSM7491) | 23S | Undetermined |
| Helicobacter fennelliae (DSM7491) | Internal control | Undetermined |
| Helicobacter cinaedi (DSM5359) | 23S | Undetermined |
| Helicobacter cinaedi (DSM5359) | Internal control | Undetermined |
| Campylobacter jejuni subsp | 23S | Undetermined |
| jejuni (DSM4688) | | |
| Campylobacter jejuni subsp | Internal control | Undetermined |
| jejuni (DSM4688) | | |
| Lactobacillus reuteri (DSM20016) | 23S | Undetermined |
| Lactobacillus reuteri (DSM20016) | Internal control | Undetermined |
| Streptococcus suis (DSM9682) | 23S | Undetermined |
| Streptococcus suis (DSM9682) | Internal control | Undetermined |
| Helicobacter pylori (HP26695) | 23S | 25.189 |
| Helicobacter pylori (HP26695) | Internal control | Undetermined |

In embodiments, the disclosure provides methods comprising quantitative PCR wherein a copy number of an H. pylori DNA segment and a copy number of a Bacteroides DNA segment present in a sample is extrapolated from a cycle threshold (Ct). The copy number is the number of copies of H. pylori genomes obtained from a sample. In quantitative PCR, a positive reaction is detected by the accumulation of a fluorescent signal. The Ct is the number of cycles required for the fluorescent signal to cross the threshold and exceed background level.

In embodiments, the Ct value may be used as a measure of the copy number of DNA segments amplified in the PCR reaction. For example, the disclosure provides reference values from samples with known H. pylori copy number, and a defined signal threshold is determined for all reactions to be analyzed. Reference copy number and Ct values according to the disclosure are presented in Table 4.

TABLE 4

| | | |
|---|---|---|
| H. pylori Genome Copies | Target Name | CT |

| | | | |
|---|---|---|---|
| 10000 | COPY | 23S rRNA | 22.676 |
| 1000 | COPY | 23S rRNA | 25.781 |
| 100 | COPY | 23S rRNA | 29.336 |
| 10 | COPY | 23S rRNA | 33.137 |
| 2 | COPY | 23S rRNA | 36.213 |
| 0 | COPY | 23S rRNA | Undetermined |

Accordingly, a fecal sample is prepared according to the embodiments of the disclosure, a quantitative PCR reaction is performed, and the number of cycles (Ct) required to reach a signal threshold is determined for the sample. The amount of H. pylori present in the sample is then determined by reference to the standards in Table 4.

In further embodiments, the disclosure provides methods for determining if H. pylori is present in a sample, and if present the relative amount of H. pylori present in the sample. Thus, the disclosure surprisingly provides threshold amounts for categorizing a fecal sample as H. pylori positive, H. pylori weakly positive, or H. pylori negative. In embodiments, the method further provides measuring the internal control level of Bacteroides DNA that controls for the total bacterial DNA obtained and extracted from a fecal sample. Accordingly, in some embodiments, the disclosure provides a method of determining if a fecal sample is H. pylori positive if a threshold level of about 5 or more copies of a H. pylori DNA segment is detected, weakly positive if a threshold level of about 2 to about 5 copies of a H. pylori gene segment is detected, or H. pylori negative if a threshold level of less than about 2 copies of a H. pylori gene segment is detected. In each case, an internal control level of about 50, about 60, about 70, about 80, about 90, about 100, about 110, about 120, about 130, about 140, about 150, or more (preferably 100) copies one or more Bacteroides DNA segments provides internal validation of the H. pylori result.

In some embodiments, a Ct of ≤about 34 is detected for a 23S rRNA DNA segment, indicating that >about 5 copies of H. pylori is present in the fecal sample (e.g., a 25 mg fecal sample) and a Ct of between about 10 to about 30 is detected in an internal Bacteroides control, wherein the sample is scored as H. pylori positive.

In other embodiments, a Ct of about 35 to about 37 is detected for a 23S rRNA DNA segment, indicating that between about 2 to about 5 copies of H. pylori is present in the fecal sample (e.g., a 25 mg fecal sample), and a Ct of between about 10 to about 30 is detected in an internal Bacteroides control, wherein the sample is scored as H. pylori weakly positive.

In still other embodiments, a Ct of more than about 37 is detected for a 23S rRNA DNA segment in a quantitative PCR reaction, indicating that less than about 2 copies of H. pylori is present in the fecal sample (e.g., a 25 mg fecal sample), and a Ct of between about 10 to about 30 is detected in an internal Bacteroides control, wherein the sample is scored as H. pylori negative.

In some embodiments, if a Ct of less than about 10 or more than about 30 is detected in an internal Bacteroides control, the sample is categorized as not reportable, and optionally another sample is obtained and the methods herein repeated until a Ct of between about 10 to about 30 is detected for the Bacteroides control.

The methods of the disclosure further provide detecting a plurality of H. pylori DNA segments in a single, multiplexed PCR reaction to increase the fidelity of the method, in each case further comprising a Bacteroides internal control. In particular embodiments, the method comprises detecting two or more of the H. pylori 23SrRNA gene, 16SrRNA gene, and Urease A gene, and in further embodiments all three of the aforementioned genes are detected in a single, quantitative PCR reaction (e.g., a multiplexed PCR reaction).

In further embodiments, the disclosure provides compositions for determining the presence of H. pylori in a sample comprising, one or a plurality of PCR primer pairs that amplify one or more H. pylori DNA segments, and one or a plurality of control PCR primer pairs that amplify one or more Bacteroides DNA segments. In further embodiments, compositions of the disclosure comprise PCR primer pairs that amplify one or more conserved H. pylori DNA segments. In embodiments, the compositions of the disclosure provide PCR primer pairs that amplify one or more H. pylori DNA segments comprising an H. pylori 23S rRNA gene, an H. pylori 16S rRNA gene, or an H. pylori Urease A gene. In particular embodiments, the disclosure provides a PCR primer pair for amplifying an H. pylori 23S rRNA gene comprising SEQ ID NO: 1 and SEQ ID NO: 2, a PCR primer pair for amplifying an H. pylori 16S rRNA gene comprising SEQ ID NO: 4 and SEQ ID NO: 5, or a PCR primer pair for amplifying an H. pylori Urease A gene comprising SEQ ID NO: 7 and SEQ ID NO: 8.

In some embodiments, the disclosure provides a kit for detecting the presence of H. pylori in a sample. In a non-limiting example, primers, enzymes for amplification and additional agents, may be comprised in a kit. The kits will thus comprise one or more of these reagents in suitable container means. The kits may also comprise agents for collection of a fecal sample, DNA extraction and isolation, purification of amplification products, labels, etc.

The components of the kits may be packaged either in aqueous media or in lyophilized form. The suitable container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the reagent containers in close confinement for commercial sale. Such containers may include injection or blow-molded plastic containers into which the desired vials are retained

In certain embodiments, kits of the disclosure comprise one or a plurality of PCR primer pairs that amplify one or more H. pylori DNA segments, and one or a plurality of PCR primer pairs that amplify one or more Bacteroides DNA segments. In embodiments, the kit comprises PCR primer pairs selected from SEQ ID NO: 1, SEQ ID NO: 2, SEQ ID NO: 4, SEQ ID NO: 5, SEQ ID NO: 7, SEQ ID NO: 8, SEQ ID NO: 10, and SEQ ID NO: 11.

In further embodiments, a kit of the disclosure comprises one or a plurality of probe sequences hybridizing with an amplification product of the one or more H. pylori DNA segments, and one or a plurality of probe sequences hybridizing with an amplification product of the one or more Bacteroides DNA segments. In embodiments, the kit comprises probe sequences selected from SEQ ID NO: 3, SEQ ID NO: 6, SEQ ID NO: 9, and SEQ ID NO: 12.

In still further embodiments, a kit of the disclosure comprises a bead homogenization suspension in a lysis buffer, wherein the lysis buffer comprises ingredients capable of breaking a bacterial cell wall, digesting protein, denaturing protein, dispersing fat, precipitating polysaccharides, or a combination thereof.

In yet further embodiments, a kit of the disclosure comprises a silica purification reagent and a DNA binding buffer. In some embodiments, the silica purification reagent comprises a silica membrane or silica beads.

In some embodiments, a kit of the disclosure further comprises a filter column comprising a filter and a silica membrane, wherein the filter column is housed within a collection vial with a closed bottom and an open top for receiving the filter column, and a wash buffer and an elution buffer. In embodiments, a kit of the disclosure comprises a PCR reaction buffer, di-nucleotide triphosphates, and one or more polymerase enzymes.

The present disclosure also provides methods of H. pylori treatment in a subject comprising obtaining a fecal sample from a subject, extracting H. pylori and Bacteroides DNA from the fecal sample, amplifying one or more H. pylori DNA segments and one or more Bacteroides DNA segments, detecting an amount of the one or more H. pylori DNA segments and an amount of the one or more Bacteroides DNA segments, comparing the amount of the one or more H. pylori DNA segments to the amount of the one or more Bacteroides DNA segments to determine the level of H. pylori in the fecal sample, and administering an H. pylori treatment if H. pylori is present at a threshold level. In some embodiments, the disclosure provides methods of H. pylori treatment in a subject, wherein treatment is administered if about 5 or more copies of an H. pylori DNA segment are detected in a fecal sample from a subject.

As used herein, the terms, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms.

In some embodiments of each or any of the above or below mentioned embodiments, the disclosure provides methods of monitoring H. pylori treatment in a subject comprising obtaining a fecal sample from the subject after administering an H. pylori treatment, extracting H. pylori and Bacteroides DNA from the fecal sample, amplifying one or more H. pylori DNA segments and one or more Bacteroides DNA segments, detecting an amount of the one or more H. pylori DNA segments and an amount of the one or more Bacteroides DNA segments, and comparing the amount of the one or more H. pylori DNA segments to the amount of the one or more Bacteroides DNA segments to determine the level of H. pylori in the fecal sample. In some embodiments, the disclosure provides methods of monitoring H. pylori treatment in a subject, wherein the fecal sample is collected at least four weeks after treatment. In some embodiments, the disclosure provides methods of monitoring H. pylori treatment in a subject, wherein the method is repeated daily, weekly, monthly, or yearly.

The present disclosure is illustrated in the following Examples, which are set forth to aid in understanding the invention, but should not be construed to limit in any way the scope of the disclosure as defined in the claims that follow.

### EXAMPLES

### Example 1: Specificity and Sensitivity of qPCR Methods

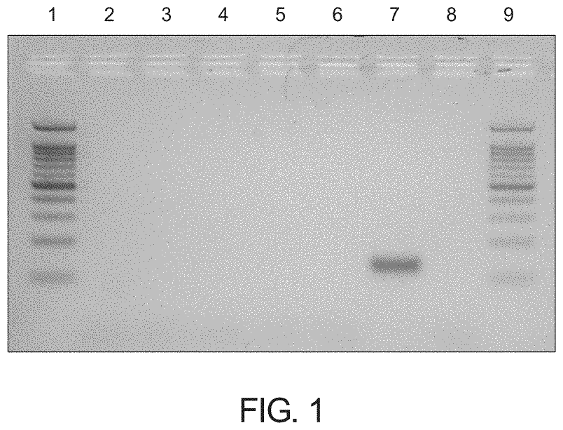

FIG. 1 shows the specificity of the disclosed methods for amplifying and detecting an H. pylori DNA segment. Bacterial genomic DNA from species closely related to H. pylori was purchased from DSMZ (Helicobacter fennelliae, Helicobacter cinaedi, Campylobacter jejuni, Lactobacillus reuteri, Streptococcus suis). H. pylori strain 26695 genomic DNA was purchased from ATCC. Bacterial genomic DNA was used as a template in PCR reactions using primers that amplify the 23S rRNA gene of pylori. PCR products were analyzed via 2% agarose gel electrophoresis. The H. pylori genomic DNA was efficiently amplified, whereas the other species showed no signal (FIG. 1).

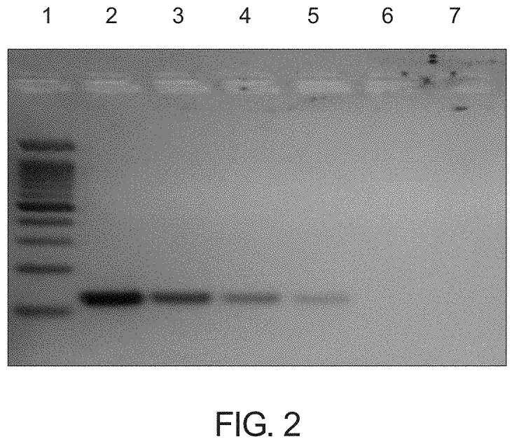

FIG. 2 and Table 4 show the sensitivity of H. pylori DNA detection using primers of the disclosure in a PCR and qPCR experiment, respectively. A series dilution was performed to prepare of H. pylori 26695 genomic DNA at 10,000 copies, 1,000 copies, 100 copies, 10 copies, and 2 copies. The dilution series genomic DNA was used in the conventional and quantitative PCR respectively amplifying the 23S rRNA gene of H. pylori 26695. For the conventional PCR, the products of PCR amplification were analyzed on agarose gel electrophoresis. Conventional PCR and agarose gel analysis show a limit of detection of 10 or more copies of H. pylori DNA, whereas real time PCR can detect 2 copies of H. pylori DNA.

### Example 2: Accuracy of the Fecal qPCR Methods of the Disclosure

Table 5 shows comparative testing data of the fecal detection methods of the disclosure in comparison to three conventional tests, two stool antigen tests and a real time PCR in gastric biopsies, in 138 matched samples. Results agreeing among the conventional tests are designated as true positive or true negative. Number 70 is the only sample that is discordant with the three tests. Fecal test qPCR test is positive, while the three conventional tests show negative.

TABLE 5

| | Sample | M-Q | | IBL | ABBOTT | Tissue |
|---|---|---|---|---|---|---|
| NO | ID | PCR | CT | ELISA | ELISA | PCR |
| 1 | MK7467 | Positive | 35.18 | Positive | Positive | Positive |
| 2 | NQ7969 | Negative | Undetermined | Negative | Negative | Negative |
| 3 | PY7665 | Negative | Undetermined | Negative | Negative | Negative |
| 4 | NA7986 | Negative | Undetermined | Negative | Negative | Negative |
| 5 | PK8019 | Negative | Undetermined | Negative | Negative | Negative |
| 6 | NKH8018 | Negative | Undetermined | Negative | Negative | Negative |
| 7 | RAL7834 | Positive | 35.60 | Positive | Positive | Positive |
| 8 | LJ7832 | Positive | 32.83 | Positive | Positive | Positive |
| 9 | PK8230 | Negative | Undetermined | Negative | Negative | Negative |
| 10 | MU8231 | Negative | Undetermined | Negative | Negative | Negative |
| 11 | PT8115 | Negative | Undetermined | Negative | Negative | Negative |
| 12 | NL8186 | Positive | 33.34 | Positive | Positive | Positive |
| 13 | NT8237 | Negative | Undetermined | Negative | Negative | Negative |
| 14 | NV8017 | Positive | 31.82 | Positive | Positive | Positive |
| 15 | NKH8018 | Negative | Undetermined | Negative | Negative | Negative |
| 16 | DKC8030 | Positive | 31.63 | Positive | Positive | Positive |
| 17 | TT8114 | Negative | Undetermined | Negative | Negative | Negative |
| 18 | MT8116 | Negative | Undetermined | Negative | Negative | Negative |
| 19 | NHD8117 | Positive | 30.69 | Positive | Positive | Positive |
| 20 | DN8235 | Negative | Undetermined | Negative | Negative | Negative |
| 21 | HN8229 | Negative | Undetermined | Negative | Negative | Negative |
| 22 | HA8239 | Positive | 34.56 | Positive | Positive | Positive |
| 23 | ML8236 | Negative | Undetermined | Negative | Negative | Negative |
| 24 | NB8232 | Positive | 35.07 | Positive | Positive | Positive |
| 25 | GN7877 | Positive | 29.92 | Positive | Positive | Positive |
| 26 | NV8240 | Positive | 29.99 | Positive | Positive | Positive |
| 27 | BP8227 | Positive | 31.61 | Positive | Positive | Positive |
| 28 | EL7998 | Negative | Undetermined | Negative | Negative | Negative |
| 29 | NH8269 | Negative | Undetermined | Negative | Negative | Negative |
| 30 | MW8302 | Negative | Undetermined | Negative | Negative | N/A |
| 31 | VB8374 | Negative | Undetermined | Negative | Negative | Negative |
| 32 | PT8187 | Negative | Undetermined | Negative | Negative | Negative |
| 33 | TN8370 | Negative | Undetermined | Negative | Negative | Negative |
| 34 | PT8367 | Negative | Undetermined | Negative | Negative | Negative |
| 35 | TN8373 | Negative | Undetermined | Negative | Negative | Negative |
| 36 | PK8364 | Negative | Undetermined | Negative | Negative | Negative |
| 37 | NY8371 | Negative | Undetermined | Negative | Negative | Negative |
| 38 | HS8377 | Positive | 34.17 | Positive | Positive | Positive |
| 39 | PT8368 | Positive | 34.44 | Positive | Positive | Positive |
| 40 | LTG8188 | Negative | Undetermined | Negative | Negative | Negative |
| 41 | NL8378 | Positive | 30.12 | Positive | Positive | Positive |
| 42 | CM7962 | Negative | Undetermined | Negative | Negative | Negative |
| 43 | LA8228 | Positive | 36.82 | Positive | Positive | Positive |
| 44 | NT8376 | Positive | 31.29 | Positive | Positive | Positive |
| 45 | OD7878 | Positive | 31.51 | Positive | Positive | Positive |
| 46 | NT8421 | Negative | Undetermined | Negative | Negative | Negative |
| 47 | HN8091 | Positive | 35.26 | Positive | Positive | Positive |
| 48 | LH8420 | Positive | 30.24 | Positive | Positive | Positive |
| 49 | LT8329 | Negative | Undetermined | Negative | Negative | Negative |
| 50 | RM8390 | Negative | Undetermined | Negative | Negative | Negative |
| 51 | NH8529 | Negative | Undetermined | Negative | Negative | Negative |
| 52 | CH8538 | Positive | 31.89 | Positive | Positive | Positive |
| 53 | LT8530 | Negative | Undetermined | Negative | Negative | Negative |
| 54 | TL8528 | Positive | 34.50 | Positive | Positive | Positive |
| 55 | OM8148 | Negative | Undetermined | Negative | Negative | Negative |
| 56 | CM8233 | Positive | 33.63 | Positive | Positive | Positive |
| 57 | VT8328 | Positive | 34.25 | Positive | Positive | Positive |
| 58 | VC8366 | Negative | Undetermined | Negative | Negative | Negative |
| 59 | DM8372 | Negative | Undetermined | Negative | Negative | Negative |
| 60 | NT8375 | Negative | Undetermined | Negative | Negative | Negative |
| 61 | KA8467 | Negative | Undetermined | Negative | Negative | Negative |
| 62 | BN8534 | Negative | Undetermined | Negative | Negative | Negative |
| 63 | NH8537 | Negative | Undetermined | Negative | Negative | Negative |
| 64 | NM8535 | Positive | 30.02 | Positive | Positive | Positive |
| 65 | RE8636 | Negative | Undetermined | Negative | Negative | Negative |
| 66 | LK8617 | Negative | Undetermined | Negative | Negative | Negative |
| 67 | LY8642 | Negative | Undetermined | Negative | Negative | Negative |
| 68 | LC8639 | Negative | Undetermined | Negative | Negative | Negative |
| 69 | PH8659 | Negative | Undetermined | Negative | Negative | Negative |
| 70 | PB8616 | Positive | 34.91 | Negative | Negative | Negative |
| 71 | MZ8724 | Negative | Undetermined | Negative | Negative | Negative |
| 72 | BM7605 | Positive | 30.71 | Positive | Positive | Positive |
| 73 | TTT8699 | Negative | Undetermined | Negative | Negative | Negative |
| 74 | TVC8692 | Negative | Undetermined | Negative | Negative | Negative |
| 75 | TT8701 | Negative | Undetermined | Negative | Negative | Negative |
| 76 | LY8642 | Negative | Undetermined | Negative | Negative | Negative |
| 77 | LNA8697 | Negative | Undetermined | Negative | Negative | Negative |
| 78 | MJ8272 | Positive | 29.50 | Positive | Positive | Positive |
| 79 | TKH8660 | Positive | 29.84 | Positive | Positive | Positive |
| 80 | VI8444 | Positive | 35.24 | Positive | Positive | Positive |
| 81 | RM-8153 | Negative | 39.76 | Negative | Negative | Negative |
| 82 | DB-8234 | Positive | 34.51 | Positive | Positive | Positive |
| 83 | VH-8661 | Positive | 31.72 | Positive | Positive | Positive |
| 84 | LK-8777 | Negative | Undetermined | Negative | Negative | Negative |
| 85 | DT-8781 | Negative | Undetermined | Negative | Negative | Negative |
| 86 | BMH-8691 | Negative | 39.27 | Negative | Negative | Negative |
| 87 | DHT8694 | Negative | Undetermined | Negative | Negative | Negative |
| 88 | DS-8334 | Positive | 32.80 | Positive | Positive | Positive |
| 89 | HP-8709 | Negative | Undetermined | Negative | Negative | Negative |
| 90 | MA-8156 | Positive | 32.00 | Positive | Positive | Positive |
| 91 | NP-8693 | Negative | 38.28 | Negative | Negative | Negative |
| 92 | NSV-8700 | Positive | 32.12 | Positive | Positive | Positive |
| 93 | RJW8152 | Negative | Undetermined | Negative | Negative | Negative |
| 94 | PT8779 | Negative | 38.62 | Negative | Negative | Negative |
| 95 | EA8455 | Negative | Undetermined | Negative | Negative | Negative |
| 96 | LJ8780 | Negative | Undetermined | Negative | Negative | Negative |
| 97 | GCM8502 | Negative | Undetermined | Negative | Negative | Negative |
| 98 | TTL8811 | Negative | Undetermined | Negative | Negative | Negative |
| 99 | CZ8631 | Negative | Undetermined | Negative | Negative | Negative |
| 100 | DK8657 | Negative | Undetermined | Negative | Negative | Negative |
| 101 | HH8912 | Negative | Undetermined | Negative | Negative | Negative |
| 102 | DH8778 | Positive | 31.50 | Positive | Positive | Positive |
| 103 | NL8914 | Negative | 38.93 | Negative | Negative | Negative |
| 104 | 8501 | Negative | Undetermined | Negative | Negative | Negative |
| 105 | NH9052 | Negative | Undetermined | Negative | Negative | Negative |
| 106 | 9004 | Negative | Undetermined | Negative | Negative | Negative |
| 107 | 9093 | Negative | Undetermined | Negative | Negative | Negative |
| 108 | 9101 | Positive | 33.55 | Positive | Positive | Positive |
| 109 | 9098 | Negative | Undetermined | Negative | Negative | Negative |
| 110 | EMM8111 | Negative | Undetermined | Negative | Negative | Negative |
| 111 | PA8275 | Negative | Undetermined | Negative | Negative | Negative |
| 112 | BO8442 | Negative | Undetermined | Negative | Negative | Negative |
| 113 | PI8460 | Negative | Undetermined | Negative | Negative | Negative |
| 114 | DS9107 | Negative | Undetermined | Negative | Negative | Negative |
| 115 | TP9111 | Negative | Undetermined | Negative | Negative | Negative |
| 116 | 9110 | Negative | Undetermined | Negative | Negative | Negative |
| 117 | HC9253 | Negative | Undetermined | Negative | Negative | Negative |
| 118 | VD9194 | Negative | Undetermined | Negative | Negative | Negative |
| 119 | GM8333 | Negative | Undetermined | Negative | Negative | Negative |
| 120 | HM8632 | Positive | 32.976 | Positive | Positive | Positive |
| 121 | LSI8501 | Negative | Undetermined | Negative | Negative | Negative |
| 122 | NH9052 | Negative | Undetermined | Negative | Negative | Negative |
| 123 | VTL9093 | Negative | Undetermined | Negative | Negative | Negative |
| 124 | PM9094 | Negative | Undetermined | Negative | Negative | Negative |
| 125 | NHM9098 | Negative | Undetermined | Negative | Negative | Negative |
| 126 | NL9101 | Positive | 34.608 | Positive | Positive | Positive |
| 127 | HN9104 | Negative | Undetermined | Negative | Negative | Negative |
| 128 | NX9113 | Negative | Undetermined | Negative | Negative | Negative |
| 129 | LV9257 | Negative | Undetermined | Negative | Negative | Negative |
| 130 | TT9255 | Positive | 32.141 | Positive | Positive | Positive |
| 131 | RMY8273 | Negative | Undetermined | Negative | Negative | Negative |
| 132 | HF8772 | Positive | 32.503 | Positive | Positive | Positive |
| 133 | TT9193 | Positive | 30.672 | Positive | Positive | Positive |
| 134 | HK9217 | Positive | 35.151 | Positive | Positive | Positive |
| 135 | HJ9219 | Positive | 32.704 | Positive | Positive | Positive |
| 136 | TM9285 | Negative | Undetermined | Negative | Negative | Negative |
| 137 | TT9286 | Negative | Undetermined | Negative | Negative | Negative |
| 138 | RE41619 | Negative | Undetermined | Negative | Negative | Negative |

Table 6 shows a statistical analysis for the 138 matched samples by comparing the fecal test of the disclosure with three conventional tests: sensitivity, specificity, positive predictive value, negative predictive value and accuracy are 100%, 98.97%, 97.67%, 100% and 99.28% respectively.

TABLE 6

| | | ELISA1 + EL1SA2 + |
|---|---|---|
| | Fecal Test | Tissue PCR |
| Positive | 43 | 42 |
| Negative | 95 | 96 |
| Total | 138 | 138 |
| M-QPCR vs | | 95% CI |
| ELISA1 + | | |
| ELISA2 2 + | | |
| Tissue PCR | | |
| Sensitivity | 100.00% | 91.59%-100% |
| Specificity | 98.97% | 94.39%-99.97% |
| Positive Predictive Value | 97.67% | 85.67%-99.66% |
| Negative Predictive Value | 100.00% | |
| Accuracy | 99.28% | 99.06%-99.98% |

### Example 32: Fecal Test PCR Amplification Efficiency and Limit of H. pylori Copy Number Detection

Table 7 shows the multiplexed quantitative PCR efficiency and cut-off for limit of H. pylori copy number detection. PCR efficiency: 23S rRNA target (102%), internal control (109%). The PCR efficiency of 100% indicates exact doubling of the copy # in each cycle of PCR. PCR efficiency of between about 80% to about 110% is considered acceptable. The Ct value of cut-off for limit of H. pylori detection is 37, converted to 2 copies of H. pylori in 25 mg of feces.

TABLE 7

| | |
|---|---|
| | Limit of H. pylori |
| | detection |
| PCR Efficiency | (23S rRNA) |

| | 23S | Internal | Cutoff |
|---|---|---|---|
| | rRNA | Control | CT = 37 |
| Fecal Sample | 102% | 109% | 2 copies |

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method of determining a level of H. pylori in a fecal sample comprising,
obtaining the fecal sample from a subject,
extracting H. pylori and Bacteroides DNA from the fecal sample;
amplifying one or more H. pylori DNA segments and one or more Bacteroides DNA segments,
detecting an amount of the one or more H. pylori DNA segments and an amount of the one or more Bacteroides DNA segments,
comparing the amount of the one or more H. pylori DNA segments to the amount of the one or more Bacteroides DNA segments to determine the level of H. pylori in the fecal sample.

2. The method of claim 1 further comprising determining whether the fecal sample is H. pylori positive, H. pylori weakly positive, or H. pylori negative.

3. The method of claim 2 further comprising determining that the fecal sample is H. pylori positive if a threshold level of about 5 or more copies of the one or more H. pylori DNA segments is detected, and a level of about 100 copies the one or more Bacteroides DNA segments is detected.

4. The method of claim 2 further comprising determining that the fecal sample is H. pylori weakly positive if a threshold level of 2-5 copies the one or more H. pylori DNA segments is detected, and a level of about 100 copies the one or more Bacteroides DNA segments is detected.

5. The method of claim 2 further comprising determining that the fecal sample is H. pylori negative if a threshold level of less than 2 copies of the one or more H. pylori DNA segments is detected, and a level of about 100 copies the one or more Bacteroides DNA segments is detected.

6. The method of claim 1, wherein the one or more H. pylori DNA segments and the one or more Bacteroides DNA segments are amplified by quantitative PCR, and wherein the amount of the one or more H. pylori DNA segments and the amount of the one or more Bacteroides DNA segments is detected using a probe sequence during a quantitative PCR reaction.

7. The method of claim 6, wherein the quantitative PCR is multiplexed.

8. The method of claim 1, wherein the one or more H. pylori DNA segments are conserved DNA segments.

9. The method of claim 1, wherein the one or more H. pylori DNA segments comprise an H. pylori 23 S rRNA gene, an H. pylori 16S rRNA gene, or an H. pylori Urease A gene.

10. The method of claim 1 further comprising amplifying each of an H. pylori 23 S rRNA gene, an H. pylori 16S rRNA gene, and an H. pylori Urease A gene.

11. The method of claim 1, wherein the Bacteroides DNA segment comprises one or more of DNA segments from Bacteroides fragilis, Bacteroides melaninogenicus, Bacteroides oralis, or a combination thereof.

12. The method of claim 1, wherein the one or more Bacteroides DNA segment is from a Bacteroides species that is present in a human regardless of age and condition.

13. The method of claim 1, wherein amplifying the one or more H. pylori DNA segments and the one or more Bacteroides DNA segments by quantitative PCR comprises,
selecting PCR primer pairs for producing amplicons comprising the one or more H. pylori DNA segments,
selecting PCR primer pairs for producing amplicons comprising the one or more Bacteroides DNA segments, and
segregating PCR primer pairs comprising one or more primers that interfere with amplicon generation by another PCR primer pair into separate PCR primer pair pools, wherein each of the separate PCR primer pair pools contain a plurality of PCR primer pairs.

14. The method of claim 1, wherein amplifying the one or more H. pylori DNA segments by quantitative PCR further comprises:
one or a plurality of PCR primer pairs selected from SEQ ID NO: 1, SEQ ID NO: 2, SEQ ID NO: 4, SEQ ID NO: 5, SEQ ID NO: 7, SEQ ID NO: 8, and
one or a plurality of probe sequences selected from SEQ ID NO: 3, SEQ ID NO: 6, and SEQ ID NO: 9.

15. The method of claim 1, wherein amplifying the one or more Bacteroides DNA segment by quantitative PCR further comprises:
one or a plurality of PCR primer pairs comprising SEQ ID NO: 10 and SEQ ID NO: 11, and
one or a plurality of probe sequences comprising SEQ ID NO: 12.

16. The method of claim 1, wherein the fecal sample is between about 0.5 grams and about 1.0 grams.

17. The method of claim 1 wherein the DNA extraction comprises bead homogenizing the fecal sample in a lysis buffer, wherein the lysis buffer comprises ingredients capable of breaking a bacterial cell wall, digesting protein, denaturing protein, dispersing fat, precipitating polysaccharides, or a combination thereof.

18. The method of claim 17, wherein the DNA extraction comprises loading the homogenized, lysed fecal sample onto a filter column comprising a filter and a silica membrane,
wherein the filter column is housed within a collection vial with a closed bottom and an open top for receiving the filter column,
forcing the soluble contents of the homogenized, lysed fecal samples through the silica membrane, and
eluting the DNA from the silica membrane.

19. A composition for determining the presence of H. pylori in a sample comprising,
one or a plurality of PCR primer pairs that amplify one or more H. pylori DNA segments, and
one or a plurality of control PCR primer pairs that amplify one or more Bacteroides DNA segments.

20. The composition of claim 19, wherein the one or more H. pylori DNA segments are conserved DNA segments.

21.-41. (canceled)


SEQUENCE LISTING (12 sequences)

SEQ ID NO: 1 - protein, 20 aa, Artificial Sequence
  Synthesized
TTCATACCAGCGTTGAAGGT

SEQ ID NO: 2 - protein, 23 aa, Artificial Sequence
  Synthesized
TTACGGCGGATACAATTCTCATA

SEQ ID NO: 3 - protein, 24 aa, Artificial Sequence
  Synthesized
ATGCTCACTTCATACCGCTCCAGC

SEQ ID NO: 4 - protein, 20 aa, Artificial Sequence
  Synthesized
AGACTAAGCCCTCCAACAAC

SEQ ID NO: 5 - protein, 19 aa, Artificial Sequence
  Synthesized
CGACCTGCTGGAACATTAC

SEQ ID NO: 6 - protein, 23 aa, Artificial Sequence
  Synthesized
CAGCTTTCGCGCAATCAGCGTCA

SEQ ID NO: 7 - protein, 21 aa, Artificial Sequence
  Synthesized
GAAGAAGCGAGAGCTGGTAAA

SEQ ID NO: 8 - protein, 24 aa, Artificial Sequence
  Synthesized
CATCAGGAAACATCGCTTCAATAC

SEQ ID NO: 9 - protein, 21 aa, Artificial Sequence
  Synthesized
TGAATTGATGCAAGAAGGGCG

SEQ ID NO: 10 - DNA, 17 bp, Artificial Sequence
  Synthesized
cagcagccgcggtaata

SEQ ID NO: 11 - protein, 21 aa, Artificial Sequence
  Synthesized
CGCAAACTTTCACAACTGACT

SEQ ID NO: 12 - DNA, 25 bp, Artificial Sequence
  Synthesized
taaagggagcgtaggtggactggta